(19) United States
(12) Patent Application Publication
(10) Pub. No.: US 2020/0405273 A1
(43) Pub. Date: Dec. 31, 2020

(54) Wearable Sensing Device

(71) Applicant: Nippon Telegraph and Telephone Corporation, Tokyo (JP)

(72) Inventors: Michiko Seyama, Tokyo (JP); Yujiro Tanaka, Tokyo (JP)

(21) Appl. No.: 16/968,659

(22) PCT Filed: Feb. 20, 2019
(86) PCT No.: PCT/JP2019/006285

§ 371 Date: Aug. 10, 2020

(30) Foreign Application Priority Data
Mar. 12, 2018 (JP) 2018-043807

(51) Int. Cl. A61B 10/00; H01M 12/04; G01N 27/333

(52) U.S. Cl. CPC A61B 10/0064; H01M 12/04; H01M 2220/30; A61B 2217/005; G01N 27/333

(57) ABSTRACT

A base material is made of a flexible resin, and formed in the shape of a sheet provided with a hollow portion. The base material can be wrapped around, for example, a forearm, an upper arm, a wrist, or the like. A sweat absorption unit is made of a plurality of fibers, and is arranged at the hollow portion for taking up sweat that has been taken in by a suction port. The sweat absorption unit is made of, for example, paper made of cellulose. A sodium ion detection electrode, a potassium ion detection electrode, and a reference electrode are allowed to contact the sweat that has been suctioned from the suction port of the sweat absorption unit and taken up into the sweat absorption unit, to detect ions contained in the sweat.

### CROSS-REFERENCE TO RELATED APPLICATIONS

This application is a national phase entry of PCT Application No. PCT/JP2019/006285, filed on Feb. 20, 2019, which claims priority to Japanese Patent Application No. 2018-043807, filed on Mar. 12, 2018, applications are hereby incorporated herein by reference.

### TECHNICAL FIELD

The present invention relates to a wearable sensing device that detects ions contained in sweat.

### BACKGROUND

Dehydration is a symptom that often appears in hyperthermia disorders commonly referred to as heat stroke. The number of heat stroke cases increases in a hot summer period, and in Japan, heat stroke occurs commonly “at home”, followed by “while at work” and “while exercising”. By gender and age, the incidence of elderly people “at home” is high, and it occurs “while exercising” in both men and women in the young generation and “while at work” in adult men who apparently tend to work in a high-temperature environment. Severe heat stroke can lead to death, and even in winter when the temperature drops, people can be dehydrated and may be taken to hospital by ambulance. Occurrence of these dehydration symptoms can be suppressed if the condition of a person can be grasped and the surrounding environment, food and drink, and the like are properly adjusted.

The reason why the incidence rate of the elderly is high indoors could be that they are in a state of being insensitive to and unable to notice by themselves changes in their body as they age. This is a problem also common to those who have impaired thermoregulatory function due to injuries of the cervical spine or the like. In addition, infants, who cannot easily express their intention relative to their own change, may have progressed dehydration without being noticed by an attendant even if there was any. For workers who are performing work, it is desirable to implement measures such as securing drinking water and cooling in an efficient manner and at a necessary timing so as not to hinder the progress of their work. Therefore, if there is a sensor device that can readily measure dehydration, the occurrence of dehydration can be prevented, making it possible to respond before a critical situation is caused.

As a common method for readily measuring whether a person is dehydrated or not, there is a technique for measuring an amount of sweat that evaporates from or flows out of the skin. This technique determines a color that appears when sweat is absorbed in order to determine an amount of water in sweat by allowing sweat to be absorbed in a filter paper or the like and by using a filter paper or the like containing dye that develops color in the presence of water. In this method, though an amount of perspiration can be grasped and intake of drinking water can be prompted according to it, it is considered difficult to identify when dehydration is actually about to occur, that is, a phenomenon in which it is difficult to perspire.

In dehydration, the index as important as the amount of sweat is the concentrations of sodium ions and potassium ions. Potassium is present as ions in human intracellular fluid, and sodium is present as ions in human extracellular fluid. In accordance with the osmotic pressure generated by the difference between the concentrations of these ions, water in the intracellular fluid travels to the extracellular fluid so that a human blood volume is generally maintained at approximately 1/13 of the body weight. When dehydration begins to occur and 10% of the blood volume is lost, the condition is considered mild dehydration, and the condition with a 30% loss thereof is said to represent dehydration potentially involving risk.

When a large amount of sweat is released in a high-temperature environment, it is difficult for a fluid to flow from the intracellular fluid to the extracellular fluid, which results in a condition of increased sodium concentration while the potassium concentration does not change. When this happens, there is no osmotic pressure difference because the salt concentration in the extracellular fluid increases, and as a result, the extracellular fluid volume does not increase, nor does the blood volume. Although the blood serves like a radiator by circulating to lower the body temperature, the effect of lowering the body temperature cannot also be obtained because the blood volume does not rise.

As a countermeasure against heat stroke as described above, there is a report with regards to ion concentration in sweat using a wearable device equipped with an ion sensor (Non-Patent Literature 2).

Note that the concentration of ions in sweat varies among different individuals, and particularly, that of sodium ions greatly differs depending on the condition of dehydration as well as the state until dehydration occurs as described above. For example, Non-Patent Literature 2 discloses that the ion concentrations in human sweat collected under a high-temperature environment from five subjects are 29-37, 56-101, 55-107, 75-117, 67-127 (mEq/L) for Na ions, and 2.8-3.7, 3.1-4.6, 3.6-5.1, 4.7-5.9, 4.4-5.3 mEq/L for K ions.

A large volume of potassium ions are present in the intracellular fluid, and though the concentration of potassium ions does fluctuate in plasma, the concentration fluctuation thereof is much smaller than that of sodium ions. Here, Eq (milliequivalent) is a unit representing an electrolytic mass, and is represented by the amount of substance (mol)×the valence of ion. Na and K are both monovalent ions, and the above unit is equivalent to the mol.

### Citation List

### Non-Patent Literature

Non-patent literature 1: W. Gao et al., “Fully integrated wearable sensor arrays for multiplexed in situ perspiration analysis”, NATURE, vol. 529, pp. 509-514, 2016.
Non-patent literature 2: L. B. Baker, et al., “Comparison of regional patch collection vs. whole body washdown for measuring sweat sodium and potassium loss during exercise”, J. Appl. Physiol., vol. 107, pp. 887-895, 2009.

### SUMMARY

### Technical Problem

Meanwhile, the concentration of ions contained in sweat depends on the concentration of ions in the blood of an individual. Therefore, even in the physiologically normal range, the concentration is expected to vary among individuals at the level of several tens of mM. From these facts, in determining dehydration as described above, it is important to measure the concentration of ions contained in sweat at the time of perspiration with accuracy. By contrast, in the technique using the above-described conventional wearable device, sweat from perspiration accumulates at a position of an ion sensor over time. For this reason, in the technique described above, what is detected is the concentration of ions of the accumulated sweat, which is different from the concentration of ions contained in the sweat produced at the time of detection.

As described above, though it is conceivable that the condition of a human body such as heat stroke can be grasped by measuring the concentration of ions contained in sweat, there is a problem in the conventional art that the concentration of ions contained in sweat at the time when sweat is produced cannot be measured with accuracy.

Embodiments of the present invention has been made to solve the above problem, and it is an object thereof to enable higher-accuracy measurement of the concentration of ions contained in sweat at the time when sweat is produced.

### Means for Solving the Problem

A wearable sensing device according to embodiments of the present invention include a sheet-like base material made of a flexible resin and provided with a hollow inside, a suction port formed on a one surface of the base material for taking in produced sweat into a hollow portion of the base material, a sweat absorption unit made of a plurality of fibers and arranged at the hollow portion for taking up the sweat that has been taken in by the suction port, a detection electrode allowed to contact the sweat that has been suctioned from the suction port and taken up into the sweat absorption unit for detecting ions contained in the sweat, a measurement chip for performing measurement of a concentration of the ions by electrochemical measurement making use of the detection electrode, and a battery serving as a power supply for the measurement chip.

In the wearable sensing device, the sweat absorption unit is made of paper.

In the wearable sensing device, a connection hole formed on an other surface of the base material is provided, the battery is a water battery, and a positive electrode of the battery is formed in contact with the sweat absorption unit through the connection hole and is allowed to contact the sweat taken up into the sweat absorption unit.

In the wearable sensing device, the detection electrode includes a sodium ion detection electrode for detecting sodium ions and a potassium ion detection electrode for detecting potassium ions.

In the wearable sensing device, the measurement chip is equipped with a communication function for transmitting a measurement result.

In the wearable sensing device, the measurement chip is formed (mounted) on the base material.

### Effects of Embodiments of the Invention

As described above, according to embodiments of the present invention, since the sweat absorption unit is provided, an excellent effect is obtained that the concentration of ions contained in sweat at the time when sweat is produced can be measured with higher accuracy.

### BRIEF DESCRIPTION OF THE DRAWINGS

FIG. 1A is a cross-sectional view showing a structure of a wearable sensing device 100 in an embodiment of the present invention.

FIG. 1B is a cross-sectional view showing a partial structure of the wearable sensing device 100 in an embodiment of the present invention.

FIG. 2 is a cross-sectional view showing a structure of the wearable sensing device 100 in an embodiment of the present invention.

### DETAILED DESCRIPTION OF ILLUSTRATIVE EMBODIMENTS

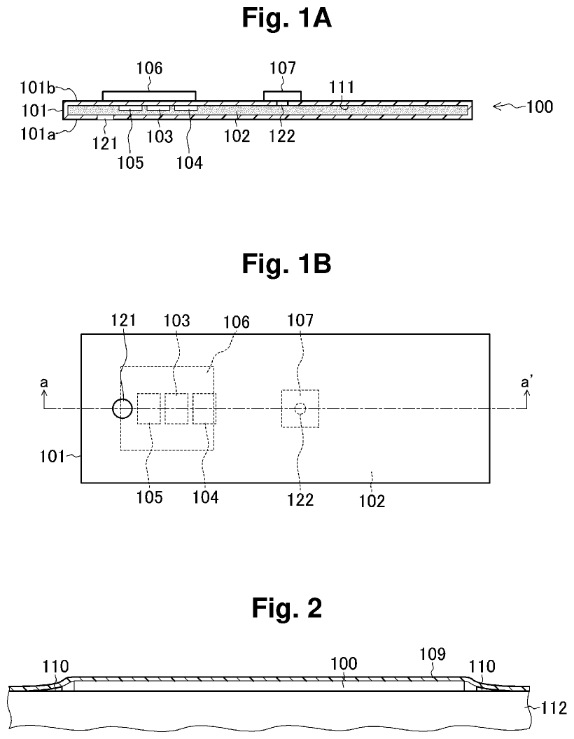

Hereinafter, a wearable sensing device 100 according to an embodiment of the present invention will be described with reference to FIGS. 1A and 1B. The wearable sensing device 100 includes a base material 101 having a hollow inside, a sweat absorption unit 102, a sodium ion detection electrode 103, a potassium ion detection electrode 104, a reference electrode 105, a measurement chip 106, and a battery 107. Note that FIG. 1A shows a cross section taken along the line a-a′ of FIG. 1B.

The base material 101 is made of a flexible resin, and is formed in the shape of a sheet provided with a hollow portion 11. The base material 101 can be wrapped around, for example, a forearm, an upper arm, a wrist, or the like. A one surface 101a of the base material 101 is a surface that contacts a skin of a human body. The base material 101 is attached to a human body by bringing the one surface 101a into contact with a skin of a human body. Further, on the one surface 101a, the base material 101 includes a suction port 121 for taking in sweat secreted from sweat glands in the skin of a human body, to which the wearable sensing device 100 is attached, into the hollow portion 11 of the base material 101.

The sweat absorption unit 102 is made of a plurality of fibers, and is arranged at the hollow portion 111 for taking up sweat that has been taken in by the suction port 121. The sweat absorption unit 102 is made of, for example, paper made of cellulose. Here, in the base material 101, preferably, a portion of the suction port 121 is hydrophilic, and the other region may be hydrophobic. Sweat secreted from sweat glands of a skin of a human body in contact with the one surface 101a is repelled on the one surface 101a, which is made hydrophobic, and is supplied continuously to the suction port 121, which is made hydrophilic.

The sodium ion detection electrode 103, the potassium ion detection electrode 104, and the reference electrode 105 are allowed to contact the sweat suctioned from the suction port 121 of the sweat absorption unit 102 and taken up by the sweat absorption unit 102. They are detection electrodes for detecting ions contained in sweat.

In the embodiment, the sweat that has reached the suction port 121 is taken up into the sweat absorption unit 102 by capillary action of the sweat absorption unit 102 that is made of paper or the like. Here, the sodium ion detection electrode 103, the potassium ion detection electrode 104, and the reference electrode 105 are formed on an inner wall of the hollow portion 11 and in contact with the sweat absorption unit 102. The sweat taken in by the suction port 121 and take up into the sweat absorption unit 102 passes through the sodium ion detection electrode 103, the potassium ion detection electrode 104, and the reference electrode 105 in contact therewith.

The sweat absorption unit 102 is preferably made of hydrophobic cellulose (for example, cellulose acetate) and hydrophilic cellulose (for example, nitrocellulose). The rate at which sweat is absorbed can be adjusted by adjusting the mixing ratio of hydrophobic cellulose and hydrophilic cellulose.

The measurement chip 106 performs measurement of the concentration of ions by known electrochemical measurement making use of the detection electrode. Further, in the embodiment, the measurement chip 106 is equipped with a communication function for transmitting a measurement result. In the embodiment, the measurement chip 106 is formed (mounted) in contact with an other surface 101b of the base material 101.

The battery 107 serves as a power supply for the measurement chip 106. The battery 107 includes, for example, a water battery. In the embodiment, the battery 107 is formed in such a way that a positive electrode using sweat (water) as an active substance is in contact with the sweat absorption unit 102 through the connection hole 122. The connection hole 122 is formed so as to penetrate the other surface 101b of the base material 101. The positive electrode of the battery 107, which is a water battery, is allowed to contact with sweat taken up into the sweat absorption unit 102 through the connection hole 122.

For example, the wearable sensing device 100 is attached to a forearm of a person to whom it is attached, and sweat is produced on the skin at the position of contact, and the generated sweat is taken in by the suction port 121. The sweat that is taken is taken up into the sweat absorption unit 102 and comes into contact with the positive electrode of the battery 107 through the connection hole 122. As a result, in the battery 107, power generation is started, and power supply to the measurement chip 106 is started.

The measurement chip 106, which has been in an operating state with the start of power supply from the battery 107, for example, transmits a connection request signal for wireless communication. The connection request signal is received, for example, by a portable terminal device not shown, resulting in a wireless connection established between the measurement chip 106 and the portable terminal device.

Furthermore, the measurement chip 106 in an operating state obtains the concentrations of sodium ions and potassium ions. The sodium ions and the potassium ions are detected when the sweat taken in by the suction port 121 has reached the sodium ion detection electrode 103, the potassium ion detection electrode 104, and the reference electrode 105. Further, the measurement chip 106 transmits an obtained value of each ion concentration to the portable terminal device.

Here, for example, by making use of information to identify a person such as a PIN (Personal Identification Number) code, authentication may be performed of the portable terminal device serving as a transmission destination. Authentication of the portable terminal device is performed by inputting the PIN code, for example, written on the base material 101 of the wearable sensing device 100.

The portable terminal device, which has received the measured value of each ion concentration, issues an alert concerning dehydration by sound, display, or the like. The portable terminal device issues the alert by comparing the value of each ion concentration with the reference concentration value or the like through operation of an installed application software.

According to the embodiment described above, the sweat that is generated by perspiration and reaches the suction port 121 is taken up into the sweat absorption unit 102. Therefore, even when a large amount of sweat is generated, it does not stay at a position of the detection electrode nor accumulate. For this reason, according to the embodiment, sweat coming into contact with the detection electrode can be limited to the sweat immediately after sweat is produced. As a result, according to the embodiment, the concentration of ions contained in sweat at the time when (immediately after) sweat is produced can be measured with higher accuracy. Furthermore, according to the embodiment, a time-series change in a component of sweat from perspiration can be observed.

### EMBODIMENT

Hereinafter, a more detailed description will be given by use of the embodiment. First, a fabrication of the wearable sensing device 100 will be briefly described. First, a support sheet made of plastic such as polydimethylsiloxane (PDMS), polyethylene terephthalate (PET), or biodegradable polylactic acid is prepared.

Next, electrode patterns made of Au are formed on an electrode formation surface of the support sheet at positions where the sodium ion detection electrode 103, the potassium ion detection electrode 104, and the reference electrode 105 are to be formed. The electrode pattern is formed by depositing Au, for example, by a sputtering method using a stencil mask or the like. The electrode pattern is formed, for example, in the vicinity of a region where the suction port 121 is arranged.

Next, a silver-silver chloride layer is formed on the formed electrode pattern. Then, the sodium ion detection electrode 103 is obtained by dropping and drying a vinyl chloride-base cocktail containing an ionophore of sodium ions onto one of the three electrode patterns. Further, the potassium ion detection electrode 104 is obtained by dropping and drying a vinyl chloride-base cocktail containing an ionophore of potassium ions onto one of the remaining two electrode patterns. The remaining one electrode pattern is the reference electrode 105. At the time when respective electrodes are thus formed, a pseudo sweat component may be brought into contact with the sodium ion detection electrode 103 and the potassium ion detection electrode 104 to condition surfaces of these detection electrodes.

Note that a surface facing the electrode formation surface of the support sheet is the other surface 101b, on which the measurement chip 106 and the battery 107 are mounted. A connection hole 122 is formed in the support sheet at a position where the battery 107 is mounted.

Next, a hollow portion forming sheet made of the above-described plastic is formed. For example, the hollow portion forming sheet is formed in the shape of a box provided with a region to serve as the hollow portion 11. Further, the suction port 121 is formed in the hollow portion forming sheet. By laminating the hollow portion forming sheet thus formed and the support sheet, the base material 101 having the hollow portion 11 is obtained.

After arranging the sweat absorption unit 102 at the hollow portion 11 to form the base material 101 having respective detection electrodes and the sweat absorption unit 102, a predetermined wiring pattern is also formed by the sputtering method or the like as mentioned above. By using a metal colloid solution, for example, to form a wiring pattern, wiring with the measurement chip 106 and the battery 107 is executed.

The base material 101 is attached to a human body by bringing the one surface 101a of the base material 101 described above into contact with a skin. When sweat generated by perspiration reaches the suction port 121, it is taken up into the sweat absorption unit 102. Then, the sweat comes into contact with the sodium ion detection electrode 103, the potassium ion detection electrode 104, and the reference electrode 105. Further, the sweat taken up into the sweat absorption unit 102 comes into contact with a positive electrode of the battery 107 through the connection hole 122.

Note that the wearable sensing device 100 may be attached to a surface of a skin 112 while being covered by a cover 109, as shown in FIG. 2. The cover 109 is patched to the surface of the skin 112 with an adhesive layer 110 provided on a periphery. Before being used, the wearable sensing device 100 is patched to a release paper not shown, and the wearable sensing device 100 is wrapped with the release paper and the cover 109.

When in use, the wearable sensing device 100 is peeled off from the release paper, and the one surface 101a is attached to a part of the skin 112 where sweat is released. After it is attached, the secreted sweat is taken up into the sweat absorption unit 102, and when it reaches the positive electrode of the battery 107 through the connection hole 122, the power supply from the battery 107 is started as described above, which makes the measurement chip 106 start operation, bringing it into a measurement state.

The produced sweat reaches the suction port 121 and is taken up into the sweat absorption unit 102, and reaches the sodium ion detection electrode 103, the potassium ion detection electrode 104, and the reference electrode 105. When the amount thereof becomes sufficient (approximately 0.7 μL), the concentration of ions contained in sweat is measurable. In this measurement, the measured ion concentration is displayed on the portable terminal device.

For example, approximately 20 minutes from the start of measurement is an adjustment time, and after the adjustment time, for example, dehydration can be determined using a measurement result. If a measured concentration of each ion deviates from a set normal state, for example, the condition is determined as dehydration. Furthermore, if a measured sodium ion concentration is equivalent to or less than 10 mEq/L, a low sodium symptom or the like is suspected and an alert thereof may be issued.

When the alert is issued, there is a risk of an abnormal state such as dehydration. Therefore, it is desired to immediately move to a cool dark place, suspend activity, consume drinking water, or the like. Furthermore, when a low sodium symptom is suspected, it is desirable to consume a beverage containing salt.

As described above, according to embodiments of the present invention, since the sweat absorption unit is provided, the concentration of ions contained in sweat at the time when sweat is produced can be measured with higher accuracy. According to the wearable sensing device of embodiments of the present invention, a risk of dehydration can be notified, for example, based on the ion concentrations of components of sweat when a person is perspiring in an active state. By doing so, not only a person to whom the wearable sensing device is attached can recognize dehydration that is being caused without being noticed by the person himself/herself, but another person who is attending or who is at a distance can also be notified of the risk of dehydration. Furthermore, by realizing quickly a risk of dehydration, preventive measures can be taken before a severe symptom develops.

Note that the present invention is not limited to the embodiment described above, and it is apparent that many modifications and combinations can be made by those skilled in the art without departing from the technical concepts of the present invention.

### REFERENCE SIGNS LIST

101 Base material
101a One surface
101b Other surface
102 Sweat absorption unit
103 Sodium ion detection electrode
104 Potassium ion detection electrode
105 Reference electrode
106 Measurement chip
107 Battery
111 Hollow portion
112 Skin 121 Suction port
122 Connection hole

1.-6. (canceled)

7. A wearable sensing device comprising:
a base material made of a flexible resin, wherein the base material comprises a hollow interior;
a suction port on a first surface of the base material, the suction port is configured to absorb sweat into the hollow interior of the base material;
a sweat absorber at the hollow interior and comprising of a plurality of fibers, the sweat absorber is configured to absorb the sweat from the suction port;
a detection electrode configured to contact the sweat that has been absorbed by the sweat absorber and configured to detect ions contained in the sweat;
a measurement chip configured to measure, by electrochemical measurement, a concentration of the ions detected by the detection electrode; and
a battery serving as a power supply for the measurement chip.

8. The wearable sensing device according to claim 7, wherein the sweat absorber is made of paper.

9. The wearable sensing device according to claim 7, further comprising:
a connection hole on a second surface of the base material, wherein the battery is a water battery, and a positive electrode of the battery is in contact with the sweat absorber through the connection hole.

10. The wearable sensing device according to claim 9, wherein the positive electrode is allowed to contact the sweat absorbed into the sweat absorber.

11. The wearable sensing device according to claim 7, wherein the detection electrode comprises:
a sodium ion detection electrode configured to detect sodium ions; and
a potassium ion detection electrode configured to detect potassium ions.

12. The wearable sensing device according to claim 7, wherein the measurement chip is equipped with a communication function for transmitting a measurement result of the measurement chip.

13. The wearable sensing device according to claim 7, wherein the measurement chip is disposed on the base material.

14. The wearable sensing device according to claim 7, wherein the base material has a sheet-like shape.

15. A method comprising:
a base material made of a flexible resin, wherein the base material comprises a hollow interior;
absorbing, by a suction port on a first surface of a base material, sweat into a hollow interior of the base material;
absorbing, by a sweat absorber at the hollow interior, absorb the sweat from the suction port, wherein the sweat absorber comprises a plurality of fibers;
contacting, by a detection electrode, the sweat that has been absorbed by the sweat absorber;
detecting, by the detection electrode, ions contained in the sweat; and
measuring, by a measurement chip and by electrochemical measurement, a concentration of the ions detected by the detection electrode.

16. The method of claim 15, wherein the base material, the suction port, the sweat absorber, the detection electrode, and the measurement chip are comprised in a wearable sensing device, and wherein the wearable sensing device further comprises a battery serving as a power supply for the measurement chip.

17. The method according to claim 16, wherein a connection hole is disposed on a second surface of the base material, wherein the battery is a water battery, and a positive electrode of the battery is in contact with the sweat absorber through the connection hole.

18. The method according to claim 17, wherein the positive electrode is allowed to contact the sweat absorbed into the sweat absorber.

19. The method according to claim 15, wherein the detection electrode comprises:
a sodium ion detection electrode configured to detect sodium ions; and
a potassium ion detection electrode configured to detect potassium ions.

20. The method according to claim 15, further comprising:
transmitting, by the measurement chip, a measurement result of the measurement chip.

21. The method according to claim 15, wherein the measurement chip is disposed on the base material.

22. The method according to claim 15, wherein the sweat absorber is made of paper.
